(19) United States
(12) Patent Application Publication
(10) Pub. No.: US 2002/0188350 A1
(43) Pub. Date: Dec. 12, 2002

(54) Annuloplasty prosthesis and a method for its manufacture

(76) Inventors: Pietro Arru, Marentino (IT); Francesco Bonetti, Roma (IT); Carla Stacchino, Torino (IT)

Correspondence Address: POPOVICH & WILES, PA, 80 SOUTH 8TH STREET, SUITE 1902, MINNEAPOLIS, MN, 55402

(21) Appl. No.: 10/164,358

(22) Filed: Jun. 5, 2002

(30) Foreign Application Priority Data
Jun. 11, 2001 (EP) 01830378.4

(51) Int. Cl. A61F 2/24

(52) U.S. Cl. 623/2.36; 623/910

(57) ABSTRACT

An annuloplasty prosthesis having at least one inner support element of elastomeric material and an outer fabric covering the support element. The elastomeric material impregnates the facing portions of the covering fabric so that the support element is fixed firmly to the covering fabric, preventing relative sliding movements between the fabric and the support element.

The present invention relates in general to a device for heart-valve repair operations and, in particular, to an annuloplasty prosthesis.

The human heart has four heart valves: the mitral valve, the tricuspid valve, the pulmonary valve, and the aortic valve. The mitral valve is situated in the left atrio-ventricular ostium and controls the unidirectionality of the blood-flow from the atrium to the ventricle. It opens during the diastole and closes during the systole, preventing the blood from flowing back from the ventricle to the atrium. Disease or genetic defects may lead to deformation or dilation of the annulus of the mitral valve, causing it to operate incorrectly, with consequent back-flow of blood. The same phenomenon may occur in the tricuspid valve which is situated between the right atrium and the right ventricle.

A method which is used to eliminate the back-flow phenomenon is that of re-establishing the correct shape and size of the valve annulus by surgical procedures known by the name of annuloplasty. Annuloplasty consists of the surgical implantation of a support prosthesis on the dilated or deformed annulus in order to re-establish its physiological size and/or shape to enable the heart valve to operate correctly. The support prostheses used in valve-repair operations take the name of annuloplasty prostheses. In most cases, a prosthesis of this type is constituted by a closed or open ring structure comprising an inner core and an outer covering of biocompatible material which enables the prosthesis to be sutured surgically.

Annuloplasty prostheses of various types have been described in the prior art. Initially, the prostheses proposed were predominantly of the rigid type in order drastically to reduce the dilation of the valve annulus. A prosthesis of this type is generally constituted by a metal core (for example, of titanium alloy or ELGILOY), an optional sheath covering the core, and an outer covering of fabric for suturing. Rigid annuloplasty prostheses are described, for example, in U.S. Pat. No. 4,055,861 by Carpentier et al., issued on Nov. 1, 1977, and U.S. Pat. No. 3,656,185 by Carpentier et al., issued on Apr. 18, 1972.

On the basis of the consideration that rigid prostheses interfere with the natural flexural movement of the annulus during the cardiac cycle, semi-rigid or fully flexible models have subsequently been proposed. Semi-rigid annuloplasty prostheses are described, for example, in U.S. Pat. Nos. 5,061,277 by Carpentier et al., issued on Oct. 29, 1991, 5,104,407 by Lam et al., issued on Apr. 14, 1992, 5,674,279 by Wright et al., issued on Oct. 7, 1997, 5,824,066 by Gross et al., issued on Oct. 20, 1998, 5,607,471 by Seguin et al., issued on Mar. 4, 1997, and 6,143,024 by Campbell et al., issued on Nov. 7, 2000. Fully flexible annuloplasty prostheses are described, for example, in U.S. Pat. Nos. 5,041,130 by Carpentier et al., issued on Aug. 20, 1991, 5,716,397 by Myers et al., issued on Feb. 10, 1998, 6,102,945 by Campbell et al., issued on Aug. 15, 2000, and 5,064,431 by Gilbertson et al., issued on Nov. 12, 1991.

Although rigid prostheses are satisfactory for some applications, they do not allow the annulus of the valve to bend along the base of the posterior cusp, with the result that they impose significant stresses on the suture points which are subjected to torsion and tension, and they prevent natural behavior of the valve. A fully flexible prosthesis follows the movements of the annulus in an optimal manner during the cardiac cycle but does not enable its shape to be reconstructed in an optimal manner. Moreover, since the above-mentioned known annuloplasty prostheses have structures in which the inner core and the outer covering are separate elements, they may cause the surgeon considerable difficulties in positioning the prosthesis and sewing it to the annulus, both due to relative sliding movements of the core and of the covering, and due to the fact that the prosthesis as a whole has a non-uniform consistency which may translate into difficulties of penetration of the needle into the prosthesis.

The present invention provides an annuloplasty prosthesis which enables the dimensions and/or the physiological shape of the annulus to be re-established without interfering with the natural flexural movement of the annulus during the cardiac cycle. The present invention provides an annuloplasty prosthesis which has improved suturability and which can be positioned and fixed more easily in the valve site of interest by sewing. The present invention achieves these advantages by means of an annuloplasty prosthesis comprising at least one inner support element of elastomeric material and an outer fabric covering the support element, the elastomeric material impregnating the facing portions of the covering fabric so that the support element is fixed firmly to the covering fabric, and preventing relative sliding movements between the fabric and the support element.

By virtue of this concept, the annuloplasty prosthesis of the present invention has a structure in which the covering fabric is fixed absolutely firmly to the support element, preventing relative movements between the support element and the covering, and facilitating the correct positioning of the prosthesis. This also permits the production of a flexible prosthesis having a particular shape which, for example, may be generally linear or circular, or may reproduce the natural shape of the annulus of a heart valve, for example, a mitral or tricuspid valve. Moreover, the annuloplasty prosthesis of the present invention has greater tear resistance and a uniform soft consistency such as to ensure improved suturability of the prosthesis in the valve site of interest. When increased stiffness properties are required, the prosthesis may also comprise a reinforcing element of material stiffer than the elastomeric material of the support element.

The invention provides a method of manufacturing an annuloplasty prosthesis as described above, comprising: (a) providing a piece of covering fabric on a support; (b) impregnating at least a portion of the outer face of the piece of fabric with elastomeric material so as to produce at least one support element of elastomeric material fixed firmly to the outer face of the fabric; (c) removing the piece of fabric from the support; (d) rolling the piece of fabric around its portion which is fixed firmly to the at least one support element so as to produce a prosthesis having at least one inner support element and an outer fabric in the form of a covering for the at least one support element; and (e) sewing the outermost free edge of the covering fabric along the edge of the prosthesis with suture thread.

The invention also provides an annuloplasty kit comprising: (1) an annuloplasty prosthesis as described above, completely or partially coated with hemocompatible carbon, preferably turbostratic carbon, and (2) a suture thread, also coated with the said carbon, for use for suturing the prosthesis to the valve annulus.

Further characteristics and advantages of the invention will become clear from the following detailed description, given purely by way of non-limiting example, with reference to the appended drawings.

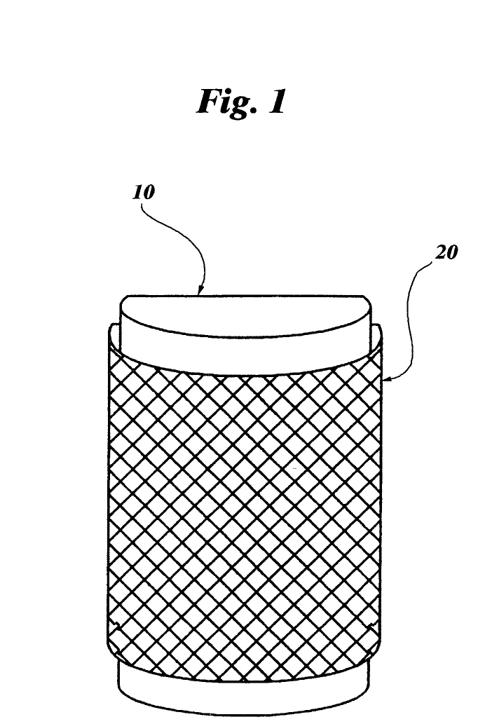

FIGS. 1 to 3 show, schematically, successive steps of the method for the manufacture of an annuloplasty prosthesis according to the invention.

FIG. 4 is a view showing an annuloplasty prosthesis according to the invention, in section.

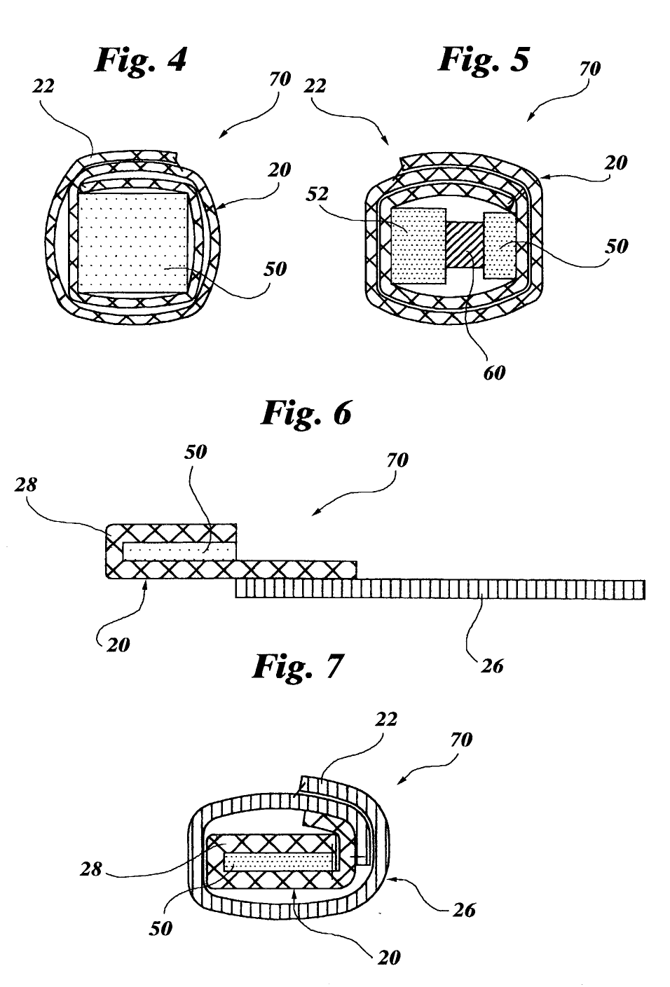

FIG. 5 is a view showing a further embodiment of the annuloplasty prosthesis according to the invention, in section.

FIG. 6 shows an intermediate stage of the method for the manufacture of a further embodiment of the annuloplasty prosthesis according to the invention.

FIG. 7 is a view showing, in section, an annuloplasty prosthesis of the type shown in FIG. 6, at an intermediate stage of the method of manufacture.

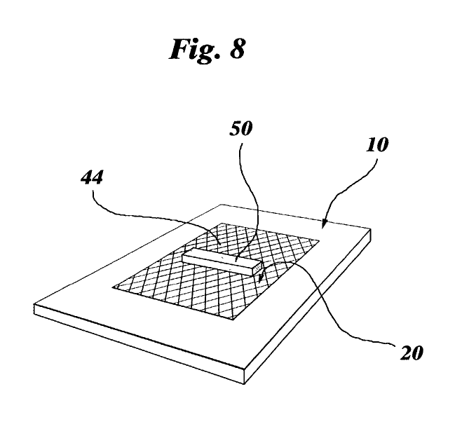

FIG. 8 shows an intermediate stage of the method of manufacturing yet another embodiment of the annuloplasty prosthesis according to the invention.

A support, which is used, in the manner of a mandrel, in a method for the manufacture of an annuloplasty prosthesis of a substantially circular shape or of a shape generally reproducing the geometry of a heart-valve annulus, is indicated 10 in FIG. 1. A piece of fabric 20, in the form of a sleeve, is fitted on the support 10. The fabric 20 is preferably made of a material selected from the group consisting of polyethylene terephthalate (PET), polytetrafluoroethylene (PTFE), polyethylene, and combinations thereof.

When the fabric sleeve 20 fitted on the support 10 is made of polyethylene terephthalate, it may advantageously be subjected to a heat treatment known as thermofixing which can produce a fabric of more homogeneous and stiffer texture such that the fabric 20 adopts the shape of the support 10 and retains it even after removal from the support. This thermofixing treatment may be performed, for example, by putting the fabric sleeve 20, fitted on the support 10, in an oven at a temperature of least 100° C. for a period of at least 20 minutes.

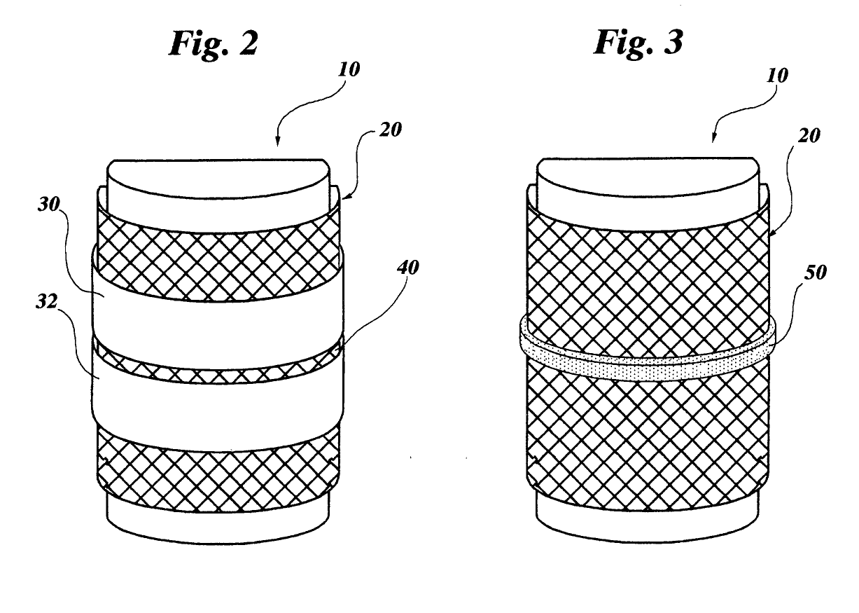

FIG. 2 shows the fabric sleeve 20 fitted on the support 10, on which in turn are fitted two guide rings, 30 and 32, respectively, which cover underlying portions of the sleeve. The guide rings 30 and 32 are spaced apart by a distance such that a strip 40 of fabric is exposed between them. The outer face of the strip 40 is impregnated with elastomeric material. After the elastomeric material has been completely polymerized, the guide rings 30 and 32 are removed so that a support element 50 of elastomeric material is produced, fixed firmly to the outer face of the fabric sleeve 20, as shown in FIG. 3.

The elastomeric material used for the support element 50 is preferably a material selected from the group consisting of silicone, polyurethane, and mixtures thereof. Barium sulphate may also be added to the elastomeric material to render the prosthesis radiopaque.

Since the support element 50 is fixed absolutely firmly to the covering fabric 20, it is possible to produce a prosthesis in which relative sliding movements between the element and the fabric 20 are prevented. This characteristic is particularly advantageous in terms of improved suturability of the prosthesis and/or of its easier positioning in the valve site of interest. Moreover, the presence of the impregnated elastomeric material in the fabric 20 gives the prosthesis a consistency such as to confer on it a particular shape which, for example, may be substantially circular or may reproduce the natural shape of a heart-valve annulus.

The fabric sleeve 20 is then slipped off the support 10, possibly cut to size, and rolled up around its portions which are fixed to the support element 50 so as to produce a prosthesis 70 having the fabric 20 as an outer covering for the inner support element 50 (FIG. 4). The outermost free edge 22 of the covering fabric 20 is sewn along the edge of the prosthesis 70 with suture thread. The end edge portion of the outermost free edge 22 may be turned towards the inside in a conventional manner, to produce a hem. The prosthesis 70 thus produced may then be thermofixed in order to shrink the yarn of the fabric.

The support 10 shown in FIGS. 1 to 3 has a generally “D”-shaped cross-section which reproduces the geometry of a mitral-valve annulus. The support 10 can therefore be used for the production of an annuloplasty prosthesis suitable for implantation in the site of a mitral valve. According to further embodiments not specifically illustrated, however, the support 10 may be of a substantially circular shape or of a shape generally reproducing the geometry of the annulus of a tricuspid valve, or of other heart valves of interest.

Annuloplasty prostheses of the type described up to now, that is, comprising a single support element 50, are flexible prostheses. However, the invention also comprises semi-rigid prosthesis as shown in FIG. 5. A prosthesis of this type comprises at least two support elements 50 and 52 and a reinforcing element 60 of material stiffer than the elastomeric material of the support elements 50, 52; this material is preferably selected from the group consisting of acetal polymers (such as, for example, polyoxymethylene), polypropylene, metal alloys such as, for example Co and Cr alloys, shape memory metals such as, for example, nitinol, and combinations thereof. Moreover, the number of support elements 50, 52 in the semi-rigid prosthesis of the invention may be selected substantially at will, in dependence on the requirements of use and on the required stiffness characteristics. The latter also determine the presence and number of reinforcing elements 60. Moreover, according to an alternative embodiment of a semi-rigid prosthesis not shown specifically, the reinforcing element 60 is not interposed between two support elements 50 and 52 but is incorporated in the elastomeric material of the element 50.

In the embodiments described up to now, the annuloplasty prosthesis has a closed-ring structure. However, the present invention also comprises embodiments not shown specifically in which the prosthesis is a ring structure which is open along one or more generatrices. Annuloplasty prostheses according to these embodiments may be produced by a method exactly the same as that described above with reference to closed rings, in which the closed ring structure produced is cut along one or more generatrices.

The present invention also includes embodiments of the prosthesis in which the outer surface of the outer covering fabric 20 (see FIGS. 4 and 5) is completely or partially coated with a thin film of hemocompatible carbon, for example, turbostratic carbon. The method for the production of the turbostratic carbon film is described, for example, in U.S. Pat. Nos. 5,084,151; 5,387,247; 5,370,684; 5,133,845; and 5,423,886. The carbon coating may be formed on the piece of fabric 20 which is subsequently rolled up to form the prosthesis 70, or directly on the finished product. Moreover, the carbon coating may be formed on the entire surface of the covering fabric 20 or may be formed selectively purely on the portion of this surface which will come into contact with the blood. The coating of the prosthesis with a thin layer of hemocompatible carbon, together with the selection of the material constituting the outer covering, contributes to improved hemocompatibility of the prosthesis and to controlled tissue growth by the receiving organism.

A prosthesis completely or partially coated with hemocompatible carbon, preferably turbostratic carbon, may advantageously be combined with a suture thread, also coated with the hemocompatible carbon, to produce an annuloplasty kit, the elements of which are characterized by a high degree of hemocompatibility.

An alternative embodiment to that described above, in which the prosthesis is completely or partially coated with hemocompatible carbon is shown in FIGS. 6 and 7. In this embodiment, a pericardium strip 26 of animal origin which may subsequently be subjected to a detoxification process, for example, as described in U.S. Pat. No. 5,873,812, is associated with the fabric sleeve 20 in the manner of an extension. FIG. 6 shows an intermediate stage of the method for the production of the annuloplasty prosthesis 70 according to this embodiment, in which a support element 50 has already been caused to adhere to the covering fabric 20 associated with the pericardium 26 of animal origin, by a method similar to that described above. In the configuration of FIG. 6, an edge portion 28 of the fabric 20 has already been wound around the support element 50, whereas the pericardium 26 is still in the extended condition. The pericardium strip 26 and the remaining portion of tissue 20 are then rolled completely around the support element 50 to adopt the configuration shown in FIG. 7, in which the pericardium 26 completely surrounds the ring. According to this embodiment, the outermost portion of the covering is therefore made of pericardium of animal origin. This embodiment has the advantage of increasing resistance to infective conditions such as endocarditis.

In the embodiments described up to now, the annuloplasty prosthesis is of a substantially circular shape or of a shape generally reproducing the geometry of a heart-valve annulus. However, other embodiments in which the prosthesis has a substantially linear structure also fall within the scope of the invention. An annuloplasty prosthesis in accordance with these embodiments may be produced from a flat piece of fabric by means of a method similar to that described above with reference to closed rings. FIG. 8 shows an intermediate stage of this method which corresponds to that shown in FIG. 3 with reference to closed loops. In FIG. 8, the outer face of a flat piece of fabric 20 disposed on a flat support 10 has already been impregnated with elastomeric material so as to produce a support element 50 fixed firmly to the outer face of the flat piece 20. The subsequent steps of the method, which are not shown specifically, are exactly the same as those described above with reference to closed rings. The flat piece of fabric 20 is removed from the support 10, possibly cut to size, and rolled around its portion which is fixed to the support element 50 so as to produce a linear structure having the covering fabric 20 as an outer covering of the inner support element 50.

Naturally, the principle of the invention remaining the same, the forms of embodiment and details of construction may be varied widely with respect to those described and illustrated purely by way of non-limiting example, without thereby departing from the scope of the invention as defined in the appended claims.

What is claimed is:

1. An annuloplasty prosthesis comprising at least one inner support element of elastomeric material and an outer fabric covering the support element, the elastomeric material impregnating the facing portions of the covering fabric so that the support element is fixed firmly to the covering fabric, and preventing relative sliding movements between the fabric and the support element.

2. An annuloplasty prosthesis according to claim 1, comprising at least one support element of elastomeric material and at least one reinforcing element.

3. An annuloplasty prosthesis according to claim 2, comprising two support elements between which a reinforcing element is interposed.

4. An annuloplasty prosthesis according to claim 2, wherein the reinforcing element is incorporated in the elastomeric material of the support element.

5. An annuloplasty prosthesis according to claim 1, wherein the elastomeric material is selected from the group consisting of silicone, polyurethane, and mixtures thereof.

6. An annuloplasty prosthesis according to claim 1, wherein the covering fabric is made of a material selected from the group consisting of polyethylene terephthalate, polytetrafluoroethylene, polyethylene, and combinations thereof.

7. An annuloplasty prosthesis according to claim 1, wherein the annuloplasty prosthesis has a substantially circular shape.

8. An annuloplasty prosthesis according to claim 1, wherein the annuloplasty prosthesis has a shape generally reproducing the geometry of a heart-valve annulus.

9. An annuloplasty prosthesis according to claim 8, wherein the annuloplasty prosthesis has a shape generally reproducing the geometry of a mitral-valve annulus.

10. An annuloplasty prosthesis according to claim 8, wherein the annuloplasty prosthesis has a shape generally reproducing the geometry of a tricuspid-valve annulus.

11. An annuloplasty prosthesis according to claim 7, wherein the annuloplasty prosthesis is a closed ring.

12. An annuloplasty prosthesis according to claim 7, wherein the annuloplasty prosthesis is a ring which is open along one or more generatrices.

13. An annuloplasty prosthesis according to claim 8, wherein the annuloplasty prosthesis is a closed ring.

14. An annuloplasty prosthesis according to claim 8, wherein the annuloplasty prosthesis is a ring which is open along one or more generatrices.

15. An annuloplasty prosthesis according to claim 1, wherein the annuloplasty prosthesis has a substantially linear shape.

16. An annuloplasty prosthesis according to claim 1, wherein at least a portion of the surface of the prosthesis is coated with hemocompatible carbon.

17. An annuloplasty prosthesis according to claim 16, wherein the hemocompatible carbon is turbostratic carbon.

18. An annuloplasty prosthesis according to claim 1, wherein pericardium of animal origin is associated with the covering fabric so as to be wrapped around it.

19. An annuloplasty kit comprising an annuloplasty prosthesis according to claim 16 and suture thread coated with hemocompatible carbon for suturing the prosthesis to the heart muscle.

20. An annuloplasty kit according to claim 19, wherein the hemocompatible carbon is turbostratic carbon.

21. A method of manufacturing an annuloplasty prosthesis according to claim 1, comprising:
(a) providing a piece of covering fabric on a support;
(b) impregnating at least a portion of the outer face of the piece of fabric with elastomeric material so as to produce at least one support element of elastomeric material fixed firmly to the outer face of the fabric;
(c) removing the piece of fabric from the support;
(d) rolling the piece of fabric around its portion which is fixed firmly to the at least one support element so as to produce a prosthesis having at least one inner support element and an outer fabric in the form of a covering for the at least one support element; and
(e) sewing the outermost free edge of the covering fabric along the edge of the prosthesis with suture thread.

22. A method according to claim 21, wherein in step (a), the piece of fabric is in the form of a sleeve fitted on the support, which has a substantially circular cross-section.

23. A method according to claim 21, wherein in step (a), the piece of fabric is in the form of a sleeve fitted on the support, which has a cross-section generally reproducing the geometry of a heart valve annulus.

24. A method according to claim 22, wherein the covering fabric is made of polyethylene terephthalate, the method further comprising, after the step (a) of fitting the fabric sleeve on the support, a step of subjecting the fabric to a heat treatment such that the fabric adopts the shape of the,support and retains it after removal from the support.

25. A method according to claim 21, wherein in step (a), the piece of fabric is in the form of a flat piece placed on the support, which is substantially flat.
